(19) United States
(12) Patent Application Publication
(10) Pub. No.: US 2026/0243670 A1
(43) Pub. Date: Aug. 20, 2026

(54) Active hyperspectral mineralogy sensing for mining

(71) Applicant: Hypermine Global Oy, Espoo (FI)

(72) Inventors: Mikhail Mekhrengin, Espoo (FI); Francisco Senna Vieira, Espoo (FI); Guillaume Genoud, Espoo (FI)

(21) Appl. No.: 19/161,732

(22) PCT Filed: Feb. 29, 2024
(86) PCT No.: PCT/FI2024/050074

§ 371 Date: Sep. 3, 2025

(30) Foreign Application Priority Data
Mar. 3, 2023 (FI) 20235253

(51) Int. Cl. G01N 21/3563; G01N 21/25; G01N 21/359; G01N 33/24; G06V 10/70

(52) U.S. Cl. CPC G01N 21/3563; G01N 21/255; G01N 21/359; G01N 33/246; G06V 10/70

(57) ABSTRACT

According to an example aspect of the present invention, there is provided a method for active hyperspectral mineralogy sensing during a Run-of-Mine in field conditions, wherein the method comprises illuminating a point on a surface of a mining material with a first broadband light signal having a range of illumination within a wavelength range of about 1350 and about 2500 nm, to enable the determination of information related to at least one water-related, carbonate-related or hydroxide-related absorbance feature, receiving a reflected version of the first broadband light signal, wherein said reflected version of the first broadband light signal is a reflection from the mining material at different wavelengths within the range of illumination of the first broadband light signal, determining a reflectance spectrum of the point of the mining material based on said reflected version of the first broadband light signal and further determining said information related to at least one water-related, carbonate-related or hydroxide-related absorbance feature based on the reflectance spectrum and performing qualitative and quantitative mineralogy analysis based on said information related to at least one water-related, carbonate-related or hydroxide-related absorbance feature.

### FIELD

Various example embodiments relate in general to mineralogical analysis and more specifically to enabling hyperspectral mineralogy sensing comprising qualitative and quantitative mineralogy analysis and mapping in the field, in mining environment, during Run-of-Mine.

### BACKGROUND

Irreplaceable natural resources play a crucial role in the sustainable development of modern society. The mission of mining companies is to meet the resource need of society keeping mining projects profitable. Mining companies face increasingly higher production costs associated with lower-grade ores due to the depletion of existing deposits and higher development complexity of new deposits. The only tenable and controllable response to this challenge is to improve recovery efficiencies. Therefore, it is desirable to apply efficient models supported by mineralogy sensing solutions for the extraction and processing of mining material.

### SUMMARY OF THE INVENTION

According to some aspects, there is provided the subject-matter of the independent claims. Some embodiments are defined in the dependent claims.

According to a first aspect of the present invention, there is provided a method for active hyperspectral mineralogy sensing during Run-of-Mine, comprising illuminating a point on a surface of a mining material with a first broadband light signal having a range of illumination within a wavelength range of about 1350 and about 2500 nm, to enable the determination of information related to at least one water-related, carbonate-related or hydroxide-related absorbance feature, receiving a reflected version of the first broadband light signal, wherein said reflected version of the first broadband light signal is a reflection from the mining material at different wavelengths within the range of illumination of the first broadband light signal, determining a reflectance spectrum of the point of the mining material based on said reflected version of the first broadband light signal and further determining said information related to at least one water-related, carbonate-related or hydroxide-related absorbance feature based on the reflectance spectrum and performing qualitative and quantitative mineralogy analysis based on said information related to at least one water-related, carbonate-related or hydroxide-related absorbance feature.

According to a second aspect of the present invention, there is provided an active hyperspectral mineralogy sensing system comprising at least one light source arranged to illuminate a point on a surface of a mining material with a first broadband light signal having a range of illumination within a wavelength range of about 1350 and about 2500 nm, to enable determination of information related to at least one water-related, carbonate-related or hydroxide-related absorbance feature, at least one spectrometer arranged to receive a reflected version of the first broadband light signal, wherein said reflected version of the first broadband light signal is a reflection from the mining material at different wavelengths within the range of illumination of the first broadband light signal and at least one processor configured to determine a reflectance spectrum of the point of the mining material based on said reflected version of the first broadband light signal, to determine said information related to at least one water-related, carbonate-related or hydroxide-related absorbance feature based on the reflectance spectrum and to perform mineralogy analysis based on said information related to at least one water-related, carbonate-related or hydroxide-related absorbance feature.

According to a third aspect of the present invention, there is provided a computer program comprising instructions which, when the program is executed by an apparatus, cause the apparatus to carry out the method of the first aspect.

### BRIEF DESCRIPTION OF THE DRAWINGS

FIG. 1 illustrates an example of a mining production flow in accordance with at least some embodiments of the present invention;

FIG. 2 illustrates a development of a mining deposit based on a mine block model in accordance with at least some embodiments of the present invention;

FIG. 3 illustrates examples of reflectance spectra of minerals with iron-related, carbonate-related and hydroxide-related absorbance features allowing for mineralogy identification, analysis and mapping in accordance with at least some embodiments of the present invention;

FIG. 4 illustrates a flowchart of a method of active hyperspectral mineralogy sensing including qualitative and quantitative mineralogy analysis and mapping in accordance with at least some embodiments of the present invention;

FIG. 5A illustrates a first example system for the implementation of active hyperspectral mineralogy sensing in accordance with at least some embodiments of the present invention;

FIG. 5B illustrates a second example system for the implementation of active hyperspectral mineralogy sensing in accordance with at least some embodiments of the present invention;

FIG. 6 illustrates an example system for the implementation of active hyperspectral mineralogy mapping in accordance with at least some embodiments of the present invention;

FIG. 7 illustrates a flowchart for qualitative and quantitative mineralogy analysis in accordance with at least some embodiments of the present invention;

FIG. 8 illustrates an example mineralogy mapping of raw material in an underground mine in accordance with at least some embodiments of the present invention;

FIG. 9 illustrates an example apparatus capable of supporting at least some embodiments of the present invention;

FIG. 10 illustrates a method in accordance with at least some embodiments of the present invention.

### EMBODIMENTS

The mining process may be represented by the following parts: mine exploration and planning; production; closure, and reclamation. Production may be divided into a Run-Of-Mine stage and a processing plant stage. The Run-of-Mine stage may comprise the mining of raw mineral material and its delivery by trucks before treatment of any sort. The processing plant stage may comprise ore extraction from raw material.

In some example embodiment of the present disclosure, sensing may be a part of the mining process. Sensing may be for example applied already during the mine exploration and planning stage. For instance, exploratory drilling, where the samples are obtained from deep beneath the surface, may be applied during the exploration stage. Once the drill core samples are collected and prepared, the samples may be analysed in a controllable environment of a laboratory with a variety of sensing methods including, for example, X-ray fluorescence (XRF) and X-ray diffraction (XRD) scanners for elemental analysis and Fourier transform infrared (FTIR) sensors and hyperspectral cameras for mineralogical analysis. The final product of the stage is a mine block model that may be based on the results of drill core analysis and represent the location and the parameters of an orebody. Next, the mine block model may guide deposit development and excavation activities during Run-Of-Mine.

Once raw material was extracted during Run-Of-Mine, it may be supplied to the processing plant. Here, another opportunity for sensing application may occur allowing ore sorting and measuring data useful for setting proper material processing parameters. At this point, the raw material may be crushed and presented as a monolayer of liberated particles allowing the application of a wide range of sensing approaches. Also, processing plants provide a controllable environment allowing for the accurate evaluation of raw material. For example, at this stage, the pneumatic sensor-based sorting machine may be exploited, which uses different sensor types, for example, XRF, FTIR, color, hyperspectral sensors, covering a vast selection of elements and minerals.

While sensing may be applied during exploration and processing activities, performing sensing during the Run-of-Mine stage may be highly challenging., e.g., if it would be desirable to perform sensing during the Run-of-Mine stage without requiring special material preparation and a controllable environment.

The set of Run-of-Mine operations may include drilling and blasting of a mine face, the extraction of the material by the shovels or underground loaders, hauling with mining trucks and separate stockpiling to manage the quality of input material to the processing plant. All the operations may be based on the input of a mine block model. The exploratory drilling of cores and their laboratory analysis may be expensive and hence, the intervals between the places or drilling may be huge and the mine block model may have limited accuracy. The lack of sensing capabilities may lead to blind spots in the Run-of-Mine stage. Without real-time sensing input, Run-of-Mine operations may produce errors. Such errors may have a significant effect on processing operations. Once the material with the wrong ore grade or mineralogical content is mixed during Run-of-Mine, it may be hard or even impossible to fix such errors.

For example, mining companies may wish to have real-time input during Run-of-Mine to improve stockpile management decisions. Run-of-Mine may be characterized by a harsh measurement environment with long measurement distances, unstable ambient lighting, and challenging moisture conditions. The sensors may need to be located outdoors at distances up to tens of meters from the raw material being extracted. Also, at least some of the mines may operate 24/7 during the night and on rainy or cloudy days. Embodiments of the present disclosure, therefore, enable active sensing technologies to deliver accurate measurement data under all possible conditions.

From the perspective of elemental sensing, active sensing may be provided for example using XRF and Laser-Induced Breakdown Spectroscopy (LIBS). Both technologies may be considered as active. XRF may apply the x-rays to the samples while LIBS may utilize a short laser pulse to create a micro-plasma on the sample surface.

Mineralogy sensing, including mineralogy identification, analysis and mapping, may detect minerals that have a negative effect on milling, metallurgical processing or occupational health, for example, talc, swelling clays, carbonates, asbestos. Also, mineralogical data may be applied for ore grade estimation based on either direct detection of ore minerals, for example, iron and nickel oxides, or alteration minerals that may be used as indicators for ore-grade information.

Having near real-time ore grade and mineralogy monitoring allows updating and improving the existing mine block model, thereby improving planning applications. For instance, ore and gangue separation during loading operations may be improved, hauling operations reducing the hauling of gangue may be optimized and the quality and the accuracy of stockpiling operations may be improved. Hence, value for all further processing operations may be achieved, to provide input to assist in the control of autonomous mining

Embodiments of the present disclosure, therefore, enable performing mineralogy analysis during Run-of-Mine. Hyperspectral data may be used to detect subtle mineralogical variations and map their distribution across surfaces of mining material resulting in a new generation of mineralogical information becoming available for mine geologists and metallurgists at the earliest stage of excavation. Mining material may be material on a surface of a mine face or excavated raw material after blasting or during hauling or stockpiling.

More specifically, embodiments of the present disclosure enable active hyperspectral mineralogy sensing. Active laser-based illumination and hyperspectral data acquisition of reflected light may be combined to enable active hyperspectral mineralogy sensing. The active illumination may be based on a set of monochromatic lasers or supercontinuum lasers, or a combination of both. In some example embodiments, collimated laser light may be exploited to allow for long-distance measurements under any ambient light conditions, to deliver robust mineralogy sensing during Run-of-Mine.

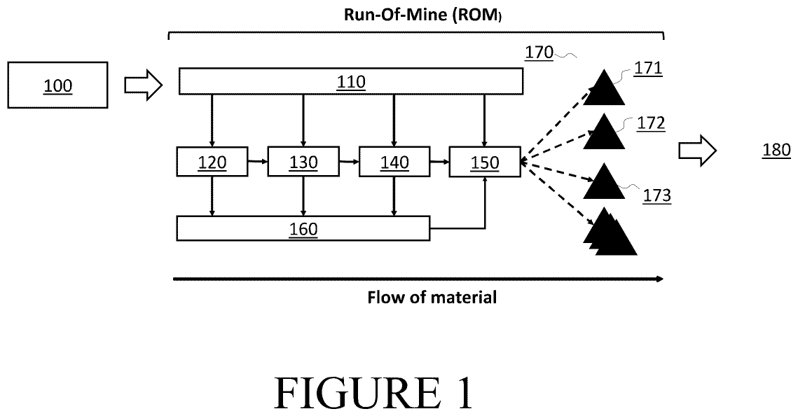

FIG. 1 illustrates an example of a mining production flow in accordance with at least some embodiments of the present invention. More specifically, FIG. 1 illustrates an example of a process, in general, describing surface and/or underground mining in accordance with at least some embodiments of the present invention. At step 100, exploratory drilling may be performed as a part of exploitation and planning activities. Next, a mine block model 110 may be developed.

Steps 120-170 may form the first part of the mining process, called Run-of-Mine. The Run-of-Mine part may comprise mining of raw mineral materials and their delivery by trucks before treatment of any sort. At step 120, the drilling and blasting of a rock face may be performed to break up the rock and make it easier to transport. A pile of rocks obtained after blasting may be referred to as a muck pile. At step 130, loading of a muck pile may be performed into trucks with shovel machines, excavators, and/or underground loaders. Then, at step 140, the hauling of a load may be performed. Steps 150, 160 and 170 may realize stockpiling process including stockpiling management, at step 150, based on a mine block model 110 and the possible mineralogy sensing data input during Run-Of-Mine, at step 160. As a result of stockpiling management at step 150, the decision on sending a hauling truck to a specific stockpile may be made. At step 170, the unloading of material from the truck to a specific stockpile 171-173 is performed. Next, raw material from the stockpiles may be moved to a processing plant 180 where a second part of the process may start. Here, multiple steps of mining material processing may be performed to extract ore and elements of interest, for example, crushing, milling, flotation, oxidization, leaching, and absorption.

The whole amount of mining material may be divided into high and low-grade material or ore (comprising the elements of interest) and waste (do not comprising economic quantities of elements of interest). Ore sorting is a crucial part of mining that includes sorting by grade, particle size, and mineralogy across many different mineral processes. In general, ore grade sorting enables the removal of gangue in the early stage of the process and saves the effort of mining companies while reducing the environmental impact.

During Run-Of-Mine, ore sorting may be implemented using stockpiling. Stockpiling may be a process, wherein mining material is separated into different stockpiles taking into account economical, technical, and geometallurgical parameters. Run-of-Mine stockpiles, formed at step 170, may be considered as essential components in the mining value chain because they can be used as temporary storage of raw material. There are at least two reasons why stockpiling might be performed. From the perspective of ore grade, this allows increasing the average grade of ore being processed by the plant to maximize the element of interest recovery. For example, the material from high-grade stockpiles may be processed first. Also, the material from the different stockpiles may be blended to ensure a certain quality of feed material. Finally, stockpiling helps to ensure a steady feed rate in case of fluctuations in raw material delivery rate. From the perspective of mineralogical content, stockpiling may provide the opportunity to divert or bypass material that is detrimental to the processing plant equipment reducing the usage of water, power, and reagents. Also, different ore-bearing minerals may require different processing flows and may be stockpiled separately. Both ore grade and mineralogical content are taken into account to decide if the processing of raw material is economically viable at the moment. At step 150, the decision on the separation of the raw material into the different stockpiles is done. This decision may be based on the mine block model and may be supported by possible mineralogy sensing data input during Run-Of-Mine, at step 160.

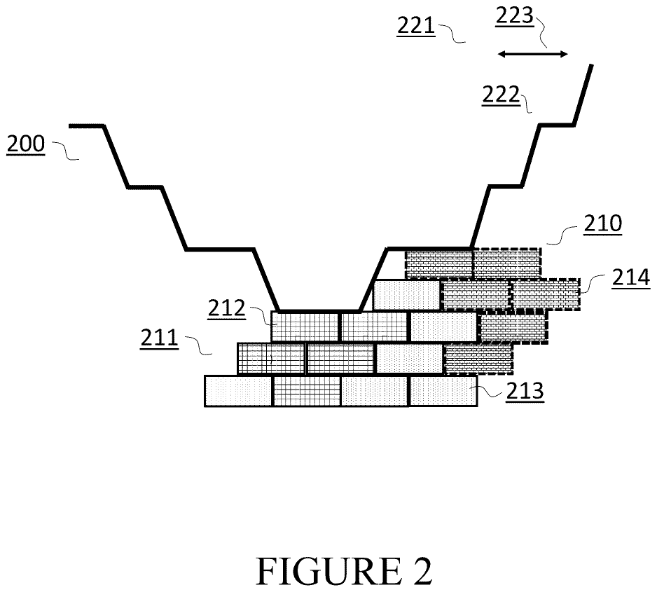

FIG. 2 illustrates a development of a mining deposit based on a mine block model in accordance with at least some embodiments of the present invention. More specifically, FIG. 2 illustrates an example of mineral deposit development using the mine block model.

The shape of a mine pit 200 may be developed according to the mine block model 210. The mine block model 210 may be a simplified representation of an ore body 211 and its surroundings shown as a stack of computer-generated cells 212-214 that represent small volumes of rock in a deposit. Ore block with high grade is denoted by 212, ore block with low grade is denoted by 213 and gangue block is denoted by 214.

Each cell may comprise estimates of data, such as element grade, density, and other geological or engineering entity values. The ore body 211 grade, location, shape, and mineralogy may be estimated before the development of a mine as a part of exploratory core drilling of 221 and 222. The exploratory core drilling and core laboratory analysis may be expensive and hence, the intervals 223 between the places or drilling may be huge reaching tens or even hundreds of meters, which would limit the mine block model resolution and the quality of stockpiling management decisions. The lack of real-time sensing input at Run-of-Mine may lead to blind spots and stockpiling errors. Such errors may have a significant effect on the processing plant operation. In some embodiments, the accuracy of the mine block model and the quality of stockpiling decisions may be improved during Run-of-Mine by real-time active hyperspectral mineralogy sensing of a muck pile of raw material directly during Run-Of-Mine 160.

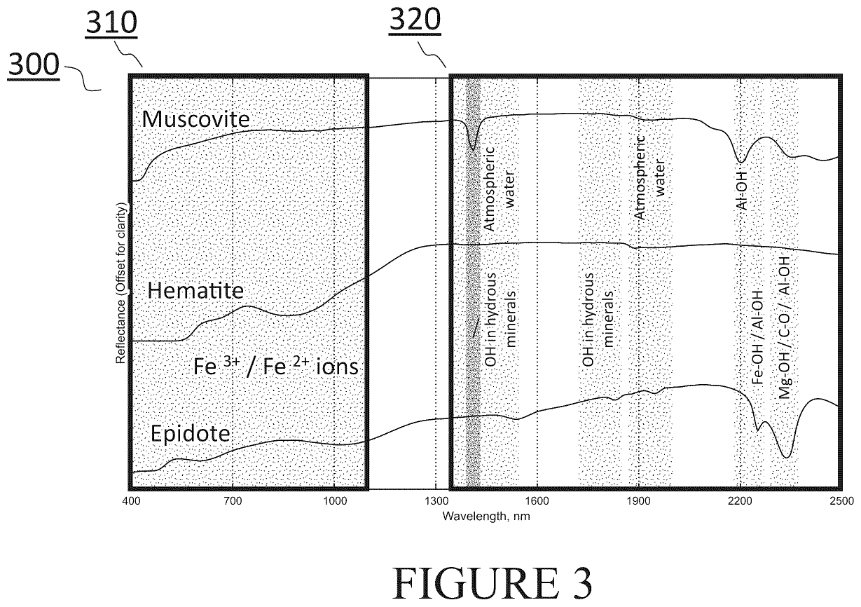

FIG. 3 illustrates examples of reflectance spectra of minerals with iron-related, water-related, carbonate-related and hydroxide-related absorbance features allowing for mineralogy identification, analysis and mapping in accordance with at least some embodiments of the present invention. More specifically, FIG. 3 illustrates examples of reflectance spectra of some minerals 300, such as muscovite, hematite, and epidote, in visible, near-infrared and short-wave infrared wavelength ranges. Hyperspectral mineralogy sensing including qualitative and quantitative mineralogy analysis may be performed using the set of absorbance features in the part of visible and near-infrared range 310 and the part of short-wave infrared range 320. The part in the visible and near-infrared ranges may be for example between about 400 and about 1100 nm, to enable the determination of information related, for example, to iron-related absorbance features. Iron-related absorbance features may be related to the absorption of iron ions and iron hydroxides. This information may be applicable for the identification of ore-bearing minerals. The part in the short-wave range may be for example between about 1350 and about 2500 nm, to enable the determination of said information related, for example, to water-related, carbonate-related and hydroxide-related absorbance features. Water-related absorbance features consist of O—H related absorption bands of free water, for example, water molecules not part of the mineral crystal structure, or hydrated minerals, for example, water molecules within the crystal structure of a mineral, for instance, CaSO₄·2H₂O. Carbonate-related absorbance features may be related to the absorption bands of C—O. Hydroxide-related absorbance features may be related to the absorption bands of Al—OH, Mg—OH, Fe—OH. This information may be applicable for the identification of gangue minerals, for example, phyllosilicates and moisture levels on a surface of mining material.

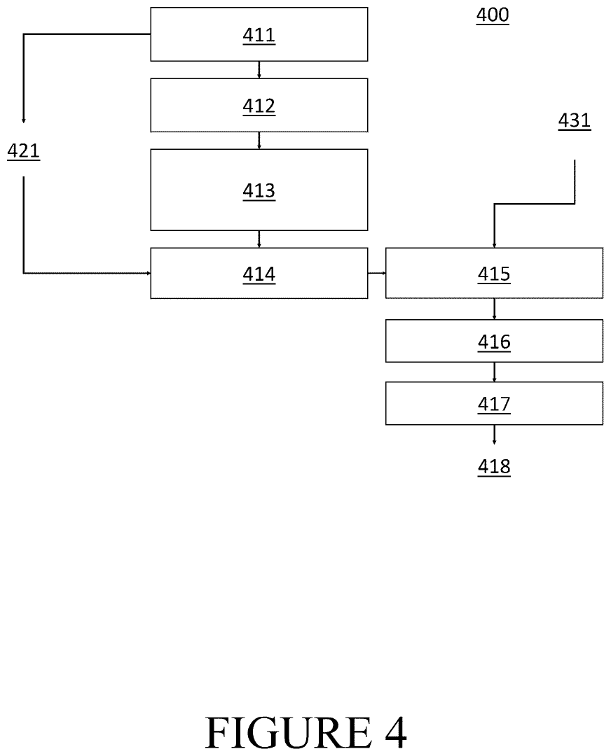

FIG. 4 illustrates a flowchart of a method of active hyperspectral mineralogy sensing including qualitative and quantitative mineralogy analysis and mapping in accordance with at least some embodiments of the present invention. Method of active hyperspectral mineralogy sensing 400 may be performed during Run-Of-Mine by the procedures described herein. More specifically, the mineralogical analysis may be performed by using active hyperspectral mineralogy sensing to enable mineralogy sensing from long distances under any ambient light conditions, e.g., in mines.

Steps 411-414 may form the first part of the method where a reflectance spectrum is captured for a single point on a surface of mining material. At step 411, a broadband light signal may be generated, e.g., using a supercontinuum laser or the set of monochromatic lasers. The application of laser light is required in active hyperspectral mineralogy sensing as the laser light can be collimated into the beam with low divergence allowing applying the illumination to mining material from long distances. For the illumination in the part of visible and near-infrared range 310 illustrated in FIG. 3, a supercontinuum laser may be applied. Alternatively, or in addition, a set of monochromatic lasers may be applied due to the extensive availability of monochromatic lasers for the visible and near-infrared wavelength ranges and the wide character of spectral features in the range 310. For the illumination in the part of short-wave infrared range 320 illustrated in FIG. 3, the application of a supercontinuum laser may be required due to limited availability of lasers with high optical power of several watts for the wavelength range of about 2300-2500 nm.

At step 412, a point on a surface of mining material may be illuminated for example at suitable times using a first broadband light signal having a range of illumination within the part of visible and near-infrared range 310, to enable determination of information related to at least one iron-related absorbance feature, or the part of short-wave infrared range 320, to enable determination of information related to at least one water-related, carbonate-related or hydroxide-related absorbance feature or the combination of both ranges 310 and 320.

At step 413, a reflected version of the broadband light signal may be acquired, the reflected version of the broadband signal being a reflection from the point of mining material at different wavelengths within the range of illumination of the broadband light signal.

At step 414, a reflectance spectrum should be determined for the point of mining material within the range of illumination of the broadband light signal. At step 414, correction of the reflectance spectrum may be performed using, for example, the prerecorded reflectance spectrum of a calibration target.

At step 415, point-by-point scanning of a plurality of points on a surface of the mining material may be performed, according to a predetermined scanning pattern, to determine the plurality of reflectance spectra for each point of mining material. That is, each point of mining face may be scanned separately. For instance, point-by-point scanning may be performed on multiple points on a surface of mining material, wherein said scanning comprises illuminating each point of the mining material with the first broadband light signal and receiving a reflected version of the first broadband light signal.

At step 416, a hyperspectral cube may be generated including the combination of all reflectance spectra and corresponding coordinates according to the predetermined scanning pattern.

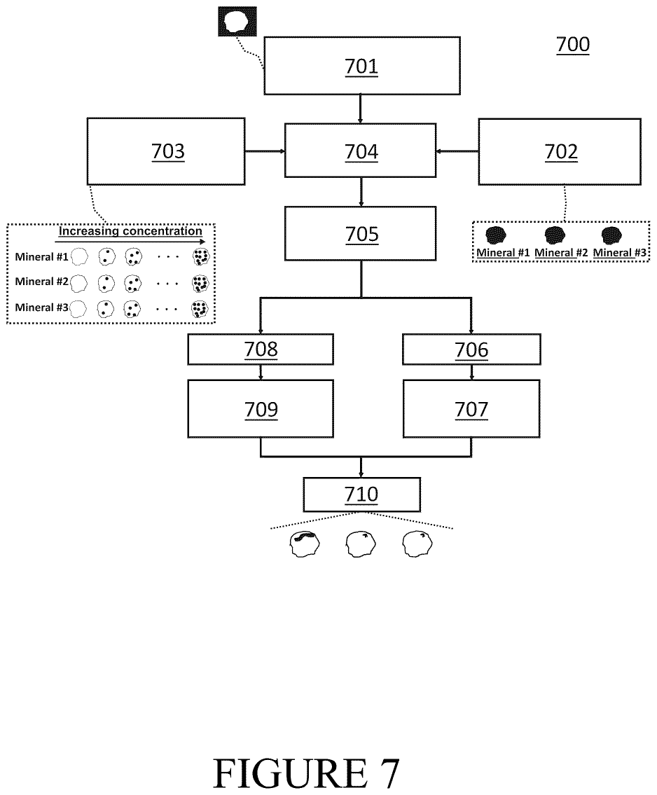

At step 417, mineral qualitative, for example, according to steps 706 and 707 illustrated in FIG. 7, and quantitative, for example, according to steps 708 and 709 illustrated in FIG. 7, analysis and mapping may be performed generating the result of mineralogy analysis including the information about the identified minerals and their concentrations as well as mineral mapping, for example, according to step 710 illustrated in FIG. 7, across the surface of mining material. At step 417, the set of methods and algorithms illustrated in FIG. 7 may be applied for mineralogy identification, concentration measurement and mapping based on at least one iron-related, water-related, carbonate-related or hydroxide-related absorbance feature. In some embodiments, the results may be presented as identified mineralogical content and average mineral concentration across the surface of the mining material by applying a pre-calibrated machine learning classifier at step 707 illustrated in FIG. 7 and a pre-calibrated quantitative model at step 709 illustrated in FIG. 7 to said hyperspectral cube and at least one iron-related, water-related, carbonate-related or hydroxide-related absorbance feature. In some embodiments, a map of a mineral distribution across the surface of the mining material may be developed at step 710 illustrated in FIG. 7 by applying a pre-calibrated machine learning classifier at step 707 illustrated in FIG. 7 and a pre-calibrated quantitative model at step 709 illustrated in FIG. 7 to said hyperspectral cube and at least one iron-related, water-related, carbonate-related or hydroxide-related absorbance feature.

At step 418, the results of the mineral qualitative and quantitative analysis may be transmitted to the mining equipment to be applied during excavation and exploration activities. In some embodiments, the identified mineralogical content and average mineral concentration across the surface of the mining material may be applied to improve loading, hauling and stockpiling operations during Run-Of-Mine and/or contribute to the mine block model. In some embodiments, the map of a mineral distribution across the surface of the mining material may be applied to improve exploration activities.

In some embodiments, a part of the broadband light signal generated at step 411 may be separated before illuminating a point on mining material 412. This part of the broadband light signal may be applied for reference measurement of a broadband light signal spectrum 421. The reference measurement spectrum may be applied as a part of the correction of the reflectance spectrum at step 414. For example, the correction may be performed by dividing the reflectance spectrum at step 414 by the reference measurement spectrum. Thus, the fluctuations in the shape of the spectrum of the broadband light signal generated at step 411 may be eliminated.

Correction of the reflectance spectrum at step 414 using the reference measurement of a broadband light signal 421 may allow taking into account deviations of the shape of spectrum of broadband light signal generated at step 411. The deviations of the shape of spectrum of broadband light signal may be caused by different environmental conditions, for example, temperature, humidity, pressure, by aging of broadband light signal. In case of application of at least one supercontinuum laser to generate broadband light signal at step 411, the reference measurement of a broadband light signal spectrum 421 may be especially important due to nonlinear nature of supercontinuum generation process which may cause fluctuations in the shape of spectrum of broadband light signal.

In some embodiments, active hyperspectral mineralogy mapping may be performed using a combination of two systems operating in a wide range of wavelengths, for example in the visible and near-infrared (400-1100 nm), and short-wave infrared (1350-2500 nm) optical ranges. Embodiments of the present invention, therefore, enable active hyperspectral mineralogy sensing including qualitative and quantitative analysis and mapping from long distances under any ambient light conditions. Such mineralogy analysis and mapping may be performed during Run-of-Mine. The system may be operated in surface mines as well as in underground mines. Mineralogy analysis and mapping may be performed using at least one illumination system based on a supercontinuum laser.

In some embodiments, the complimentary measurements of distance information using Light Detection and Ranging (LIDAR), humidity and/or pressure 431 may be performed and applied for corrections at step 417.

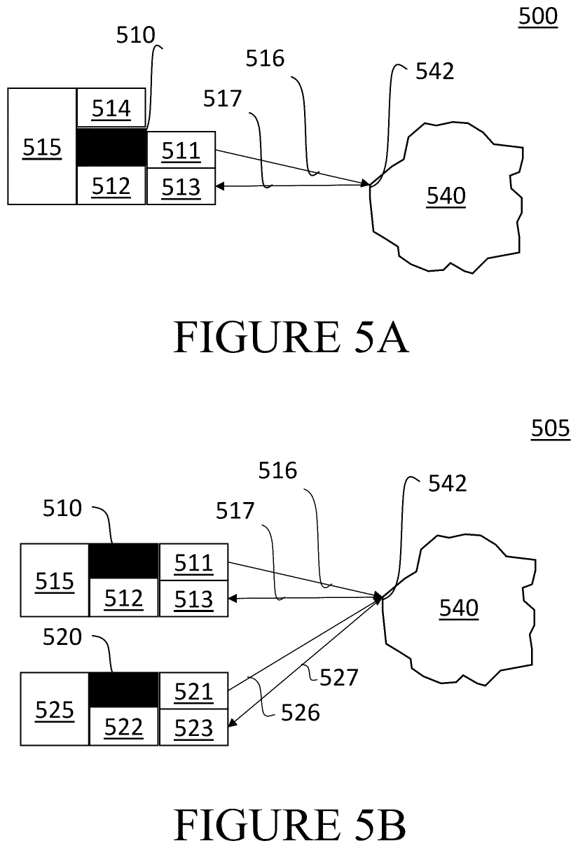

FIG. 5A illustrates a first example system for the implementation of active hyperspectral mineralogy sensing in accordance with at least some embodiments of the present invention. Exemplary active hyperspectral mineralogy sensing system 500 may comprise at least one light source 510, at least one spectrometer 512, at least one optical illumination scheme 511, at least one light collection optical scheme 513 and at least one apparatus 515. The active hyperspectral mineralogy sensing system 500 may be used to illuminate and determine mineralogy information about mining material 540, and more specifically about point 542.

Apparatus 515 may be a processing unit or data processing board containing Application-Specific Integrated Circuit (ASIC), System-on-Chip (SoC), Field-Programmable Gate Array (FPGA), Single Board Computer (SBC) or microprocessor for example and be configured to control at least one light source 510 and at least one spectrometer 512. Apparatus 515 may be connected to a light source 510 and/or a spectrometer 512 via wired connections respectively. Apparatus 515 may be, in general, a control device. Apparatus 515 may be means for performing method steps illustrated on FIG. 4 and FIG. 7.

For instance, at least one processor of apparatus 515 may be configured to cause illumination of point 542 by first broadband light source 510. At least one processor of apparatus 515 may be configured to cause said illumination by transmitting a command to the first broadband light source 510, to enable dynamic operation and power savings when there is no need to illuminate the target.

The command may be a command that indicates to first broadband light source 510 that it needs to switch on its light generation, e.g., for a certain time period. Saving of power is beneficial in particular for applications, wherein it is not possible to provide a connection to a fixed electric power network for active hyperspectral mineralogy sensing system 500. For example in mines battery-powered devices may be used, as the mines are often in remote locations, wherein it is not possible, or reasonable, to provide the connection to the fixed electric power network. Hence, power-efficient solutions are preferred.

In some embodiments, a supercontinuum laser may be used as the first light source 510. An application of supercontinuum laser as a light source 510 for illumination of a point 542 of mine face 540 allows having only one optical source for the wide range of wavelengths, for example, about 1350 and about 2500 nm. At the same time, the light of supercontinuum laser may be collimated allowing for remote illumination up to hundreds of meters. Moreover, supercontinuum lasers may be characterized by high optical power of several watts. All these parameters are important for remote sensing in mining applications.

The first light source 510 may be arranged to illuminate a point 542 of a mine face 540 with the first broadband light signal 516. First broadband light signal 516 may have a range of illumination within the part of short-wave infrared range 320 illustrated in FIG. 3.

First spectrometer 512 may be arranged to receive reflected version 517 of first broadband light signal 516, wherein reflected version 517 of first broadband light signal 516 is a reflection from point 542 of mining material 540 at different wavelengths within the range of illumination of first broadband light signal 516. For instance, at least one processor of apparatus 515 may be configured to cause reception of reflected version 517 of first broadband light signal 516. At least one processor of apparatus 515 may be configured to cause said reception by transmitting a command to first spectrometer 512 to enable dynamic operation and further power savings when there is no need to receive from the target. The command may be a command that indicates to first spectrometer 512 that it needs to switch on its light acquisition.

Optical scheme 511 may be applied to collimate or focus the first broadband light signal 516 of the first broadband light source 510 allowing delivering the first broadband light signal 516 to point 542 of mining material 540 from a long distance, for example, from 2 to 30 meters. Optical scheme 513 may be applied to collect reflected version 517 of the first broadband light signal 516 from point 542 of mining material 540 at different wavelengths within the range of illumination of the first broadband light signal 516.

Apparatus 515 may be configured to determine a reflectance spectrum of point 542 of mine face or raw material 540 based on reflected version 517 of first broadband light signal 516 and to determine said information related to at least one water-related, carbonate-related or hydroxide-related absorbance features based on the reflectance spectrum. For instance, at least one processor of apparatus 515 may be configured to determine a reflectance spectrum of point 542 of mine face or raw material 540 based on reflected version 517 of first broadband light signal 516 and further determine said information related to at least one water-related, carbonate-related or hydroxide-related absorbance feature based on the reflectance spectrum.

Apparatus 515 may be configured to perform qualitative and quantitative analysis of mineralogy for a single point 542 of mining material 540 using the set of methods and algorithms illustrated in FIG. 7.

In some embodiments, the active hyperspectral mineralogy sensing system 500 may comprise spectrometer 514 for reference measurement of the spectrum of a part of first broadband light signal 516 according to step 421 in FIG. 4. The reference measurement spectrum may be applied as a part of the correction of the received reflectance spectrum of reflected light 517 in apparatus 515 according to step 414 in FIG. 4.

FIG. 5B illustrates a second example system for the implementation of active hyperspectral mineralogy sensing in accordance with at least some embodiments of the present invention. Hyperspectral mineralogy sensing system 505 illustrated in FIG. 5B may comprise a second broadband light source 520, second spectrometer 522, second apparatus 525 and optical schemes 521, 523 to collimate or focus the second broadband light signal 526 and collect reflected version 527 of second broadband light signal 526 from point 542 of mining material 540 at different wavelengths within the range of illumination of second broadband light signal 526.

Second broadband light source 520 may be arranged to illuminate point 542 of mine face or raw material 540 with second broadband light signal 526 having a range of illumination within in the part of visible and near-infrared range 310. The part of visible and near-infrared ranges may be between about 400 and about 1100 nm, e.g., between about 400 and about 1100 nm. In some embodiments, supercontinuum illumination may be used. In some embodiments, second broadband light source 520 may comprise a monochromatic laser, to operate within the visible and near-infrared range. That is, active hyperspectral mineralogy sensing system 505 may operate in the visible and near-infrared range using the monochromatic laser. In some embodiments, hyperspectral mineralogy sensing system 505 may operate in the visible and near-infrared range using a set of monochromatic lasers.

Second spectrometer 522 may be arranged to receive reflected version 527 of second broadband light signal 526, wherein reflected version 527 of second broadband light signal 526 is a reflection from point 542 of mine face or raw material 540 at different wavelengths within the range of illumination of second broadband light signal 526. Apparatus 525 may be configured to determine a reflectance spectrum of point 542 of mine face or raw material 540 based on reflected version 527 of second broadband light signal 526 and to determine said information related to iron-related absorbance features based on the reflectance spectrum.

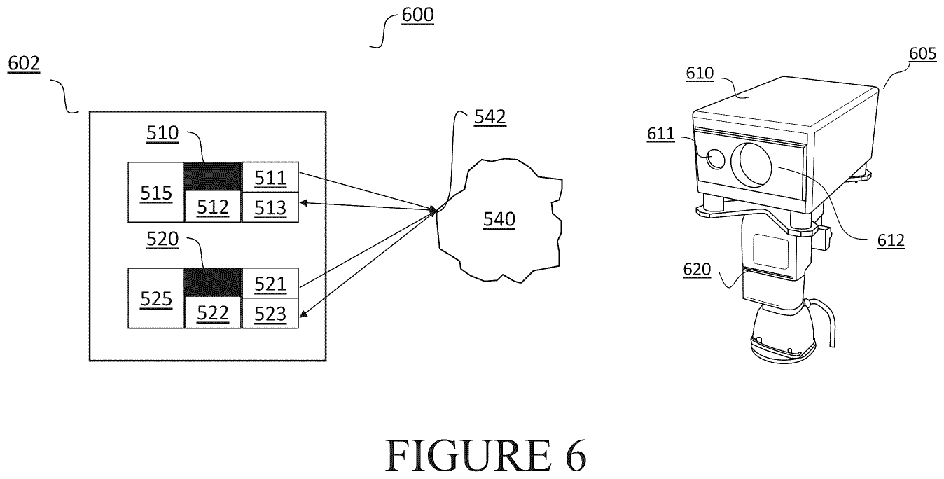

FIG. 6 illustrates an example system for the implementation of active hyperspectral mineralogy mapping in accordance with at least some embodiments of the present invention. System 600 may be applied for spatial scanning a plurality of points on mining material 540. System 600 on the left in FIG. 6 comprises, in addition to parts of system 505 in FIG. 5B, a pan-tilt unit 602 to perform two-dimensional scanning of mining material 540. Steps 411-414 of the method may be repeated for a plurality of points on mine face or raw material 540 and a plurality of reflectance spectra and coordinates according to predetermined scanning pattern may be fused for a plurality of points to generate a hyperspectral cube for mine face or raw material 540.

FIG. 6 illustrates on the right an example of the realisation of a 2D scanning active hyperspectral mineralogy mapping system 605 for spatial scanning of a plurality of points on mine face or raw material 540. System 605 comprises active hyperspectral mineralogy sensing unit 610 containing at least one light source, at least one spectrometer, at least one optical scheme to collimate or focus a broadband light signal, at least one optical scheme to collect the reflected version of a broadband light signal from point of mining material at different wavelengths within the range of illumination of a broadband light signal. Optical schemes are protected with optical windows 611, 612. In some embodiments, optical schemes could have one optical axis and may be protected using one optical window. System 605 comprises a pan-tilt unit 620 to move active hyperspectral mineralogy sensing unit 610 in pan and tilt directions and perform spatial scanning of a plurality of points on a surface of mine face or raw material and generate a hyperspectral cube.

FIG. 7 illustrates a flowchart for qualitative and quantitative mineralogy analysis 700 in accordance with at least some embodiments of the present invention. The system 600 from FIG. 6 may acquire, at step 701, hyperspectral cubes containing reflectance spectra from a plurality of points from mining material. The two dimensions of the hyperspectral cube represent the surface of mining material and the third dimension represents a reflectance spectrum. As a reference for qualitative analysis, the hyperspectral cubes comprising reflectance spectra from a plurality of points from pure minerals may be pre-recorded, at step 702, with the system 600. As a reference for quantitative analysis, the hyperspectral cubes comprising reflectance spectra from a plurality of points of reference mineral samples with known concentrations may be pre-recorded, at step 703, with the system 600.

The set of hyperspectral cubes 701, 702, 703 may be submitted to a background removal algorithm 704, for example, setting a threshold for the pixel scores of Principal Component Analysis (PCA), for selection of the Region of Interest (ROI) as well as removal of reflectance spectra of outlier, for example, spurious materials on the rock surface, edges, and bad points, for example, regions of detector saturation. The reflectance spectra of points contained in the ROI may be submitted to spectral smoothing, for example, Savitzky-Golay, and baseline correction algorithms 705, for example, Asymmetric Least Squares Baseline Correction, to minimize noise and spectral baseline distortions caused by variations in the sample geometry and surface roughness.

In some embodiments, qualitative analysis may be performed. For qualitative analysis, a machine learning classifier, for example, Nearest Neighbors (k-NN), may be calibrated at step 706 using the corrected, at step 705, reflectance spectral from hyperspectral cubes containing reflectance spectra of pure minerals obtained at step 702. Afterwards, the reflectance spectrum of each point in the corrected hyperspectral cube of a surface of mine face or raw material 701 may be assigned to one or several minerals from the set of pure minerals 702 by applying the machine learning classifier at step 707. At step 710, a qualitative mineral map may be generated based on coordinates from the hyperspectral cube. Qualitative mapping might not require prior information about the unknown samples and may be used for exploration purposes, for example, screening which minerals are present in a location and improving the mine block model.

In some embodiments, quantitative analysis may be performed. For quantitative mineral mapping, the hyperspectral cubes containing reflectance spectra of reference mineral samples with known concentrations of the target minerals (mineral #1, mineral #2, mineral #3) 703 may be used to calibrate one or more univariate (peak absorbance value at a single wavelength) or multivariate quantitative models at step 708, for example, Partial Least Squares (PLS). The concentrations of all target minerals (mineral #1, mineral #2, mineral #3) in every point in the corrected hyperspectral cube of a surface of mine face or raw material 701 may be predicted by applying the quantitative model 709, thus generating a quantitative distribution map. At step 710, a quantitative mineral map may be generated. Quantitative mapping may require prior information about the mineralogical content in a mining location and may be used for estimation of the concentration of specific minerals of interest for excavation purposes, for example, supporting stockpiling decisions and improving the accuracy of the mine block model during Run-of-Mine.

In some embodiments, systems 500 and 505 from FIG. 5 or 600 and 605 from FIG. 6 may acquire a reflectance spectrum from a single point on the surface of mining material. Then, the qualitative and quantitative mineralogy analysis may be performed for a single point using the combination of steps 705-709 and reference spectra acquired at steps 702 and 703.

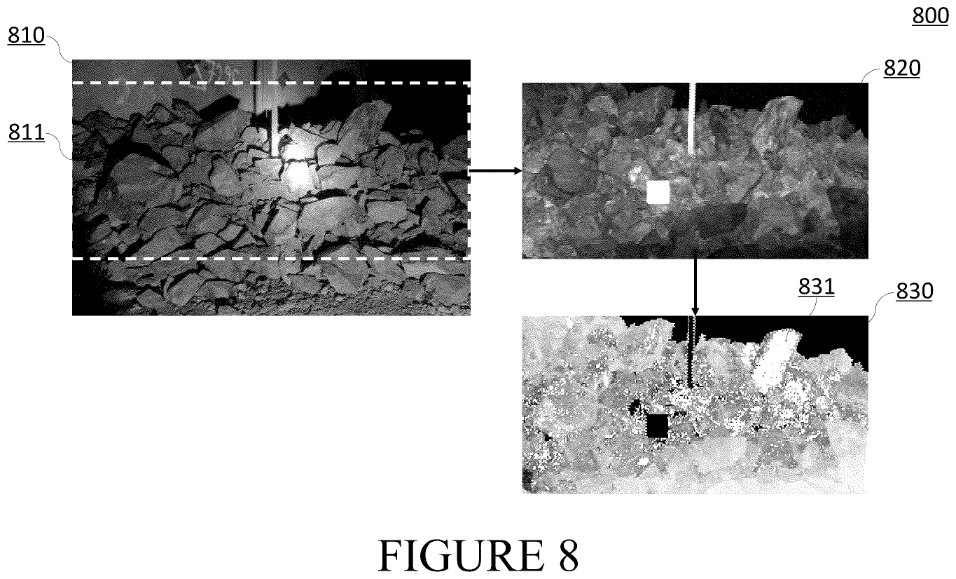

FIG. 8 illustrates an example of mineralogy mapping of raw material in an underground mine 800 in accordance with at least some embodiments of the present invention. Raw material according to photo 810 may be located at a mining location in an underground mine. The part of a surface of raw material 811 may be remotely scanned using active hyperspectral mineralogy mapping system 600 according to steps 411-416. The acquired hyperspectral cubes containing reflectance spectra from a plurality of points of raw material may be presented as a false-color image 820 and further the mineralogical information 831 may be superimposed on the false-color picture forming mineral map 830.

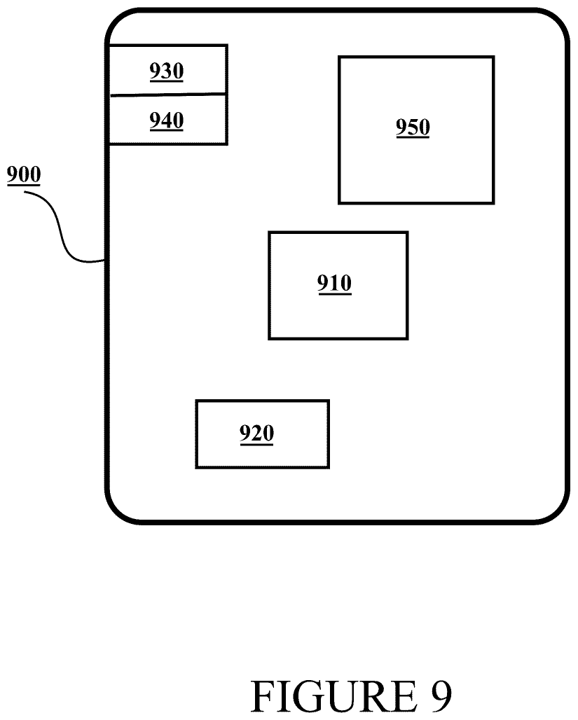

FIG. 9 illustrates an example apparatus capable of supporting at least some embodiments of the present invention. Illustrated is apparatus 900, which may comprise or be apparatus 515 or apparatus 525 of FIGS. 5A and 5B.

Comprised in apparatus 900 may be processing unit, i.e., processing element, 910, which may further comprise, for example, a single-or multi-core processor wherein a single-core processor comprises one processing core and a multi-core processor comprises more than one processing core. Processing unit 910 may comprise, in general, a control device. Processing unit 910 may comprise one or more processors. Processing unit 910 may be a control device. Processing unit 910 may comprise at least one Application-Specific Integrated Circuit, ASIC. Processing unit 910 may comprise at least one Field-Programmable Gate Array, FPGA. Processing unit 910 may comprise at least one Single Board Computer, SBC. Processing unit 910 may be means for performing method steps in apparatus 900. Processing unit 910 may be configured, at least in part by computer instructions, to perform actions.

Apparatus 900 may comprise memory 920. Memory 920 may comprise Random-Access Memory, RAM, and/or permanent memory. Memory 920 may comprise at least one RAM chip. Memory 920 may comprise solid-state, magnetic, optical and/or holographic memory, for example. Memory 920 may be at least in part accessible to processing unit 910. Memory 920 may be at least in part comprised in processing unit 910. Memory 920 may be means for storing information, such as a phase and amplitude of a reflected signal. Memory 920 may comprise computer instructions that processing unit 910 is configured to execute. When computer instructions configured to cause processing unit 910 to perform certain actions are stored in memory 920, and apparatus 900 overall is configured to run under the direction of processing unit 910 using computer instructions from memory 920, processing unit 910 and/or its at least one processing core may be considered to be configured to perform said certain actions. Memory 920 may be at least in part comprised in processing unit 910. Memory 920 may be at least in part external to apparatus 900 but accessible to apparatus 900.

Apparatus 900 may comprise a wired and/or wireless transmitter 930. For instance, the transmitter 930 may comprise at least one transmit antenna, or the transmitter 930 may be connectable to the at least one transmit antenna. If apparatus 900 is a control device for at least one light source 510, 520 and/or at least one spectrometer 512, 522 the transmitter 930 may be connectable to at least one light source 510, 520 and/or at least one spectrometer 512, 522.

Apparatus 900 may also comprise a wired and/or wireless receiver 940. For instance, the receiver 940 may comprise at least one receive antenna, or the receiver may be connectable to the at least one receive antennas. If apparatus 900 is a control device for at least one light source 510, 520 and/or at least one spectrometer 512, 522, the receiver 940 may be connectable to at least one light source 510, 520 and/or at least one spectrometer 512, 522.

Apparatus 900 may also comprise a user interface, UI, 950. UI 950 may be web user interface and may be accessible via wired or wireless connection. A user may be able to operate apparatus 900 via UI 950. Also, UI 950 may be used for displaying information to the user. For example, UI 950 may be used for providing, i.e., displaying, determined information, like results of mineralogy identification, analysis and mapping.

Processing unit 910 may be furnished with a transmitter arranged to output information from processing unit 910, via electrical leads internal to apparatus 900, to other devices comprised in apparatus 900. Such a transmitter may comprise a serial bus transmitter arranged to, for example, output information via at least one electrical lead to memory 920 for storage therein. Alternatively to a serial bus, the transmitter may comprise a parallel bus transmitter. Likewise processing unit 910 may comprise a receiver arranged to receive information in processing unit 910, via electrical leads internal to apparatus 900, from other devices comprised in apparatus 900. Such a receiver may comprise a serial bus receiver arranged to, for example, receive information via at least one electrical lead from receiver 940 for processing in processing unit 910. Alternatively to a serial bus, the receiver may comprise a parallel bus receiver.

Processing unit 910, memory 920, transmitter 930, receiver 940 and/or UI 950 may be interconnected by electrical leads internal to apparatus 900 in a multitude of different ways. For example, each of the aforementioned devices may be separately connected to a master bus internal to apparatus 900, to allow for the devices to exchange information. However, as the skilled person will appreciate, this is only one example and depending on the embodiment various ways of interconnecting at least two of the aforementioned devices may be selected without departing from the scope of the present invention.

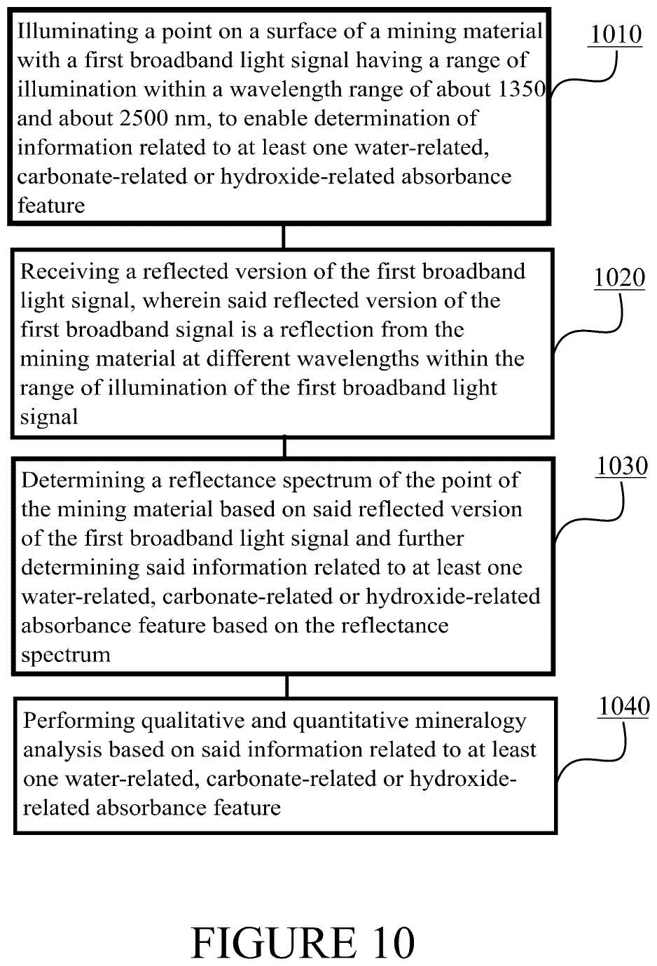

FIG. 10 illustrates a method in accordance with at least some embodiments of the present invention. The method may be for active hyperspectral mineralogy sensing during a Run-of-Mine in field conditions.

The method may comprise, at step 1010, illuminating a point on a surface of a mining material with a first broadband light signal having a range of illumination within a wavelength range of about 1350 and about 2500 nm, to enable the determination of information related to at least one water-related, carbonate-related or hydroxide-related absorbance feature. The method may also comprise, at step 1020, receiving a reflected version of the first broadband light signal, wherein said reflected version of the first broadband light signal is a reflection from the mining material at different wavelengths within the range of illumination of the first broadband light signal. The method may further comprise, at step 1030, determining a reflectance spectrum of the point of the mining material based on said reflected version of the first broadband light signal and further determining said information related to at least one water-related, carbonate-related or hydroxide-related absorbance feature based on the reflectance spectrum. Finally, the method may comprise, at step 1040, performing qualitative and quantitative mineralogy analysis based on said information related to at least one water-related, carbonate-related or hydroxide-related absorbance feature.

It is to be understood that the embodiments of the invention disclosed are not limited to the particular structures, process steps, or materials disclosed herein, but are extended to equivalents thereof as would be recognized by those ordinarily skilled in the relevant arts. It should also be understood that terminology employed herein is used for the purpose of describing particular embodiments only and is not intended to be limiting.

Reference throughout this specification to one embodiment or an embodiment means that a particular feature, structure, or characteristic described in connection with the embodiment is included in at least one embodiment of the present invention. Thus, appearances of the phrases “in one embodiment” or “in an embodiment” in various places throughout this specification are not necessarily all referring to the same embodiment. Where reference is made to a numerical value using a term such as, for example, about or substantially, the exact numerical value is also disclosed.

As used herein, a plurality of items, structural elements, compositional elements, and/or materials may be presented in a common list for convenience. However, these lists should be construed as though each member of the list is individually identified as a separate and unique member. Thus, no individual member of such list should be construed as a de facto equivalent of any other member of the same list solely based on their presentation in a common group without indications to the contrary. In addition, various embodiments and example of the present invention may be referred to herein along with alternatives for the various components thereof. It is understood that such embodiments, examples, and alternatives are not to be construed as de facto equivalents of one another, but are to be considered as separate and autonomous representations of the present invention.

In some embodiments, a computer program may be configured to cause a method in accordance with the embodiments described above and any combination thereof. In some embodiments, a computer program product, embodied on a non-transitory computer readable medium, may be configured to control a processing unit to perform a process comprising the embodiments described above and any combination thereof.

In some embodiments, an apparatus, such as, for example, apparatus 150, may comprise at least one processing unit, and at least one memory including computer program code, wherein the at least one memory and the computer program code are configured to, with the at least one processing unit, cause the apparatus at least to perform the embodiments described above and any combination thereof.

Furthermore, the described features, structures, or characteristics may be combined in any suitable manner in one or more embodiments. In the preceding description, numerous specific details are provided, such as examples of lengths, widths, shapes, etc., to provide a thorough understanding of embodiments of the invention. One skilled in the relevant art will recognize, however, that the invention can be practiced without one or more of the specific details, or with other methods, components, materials, etc. In other instances, well-known structures, materials, or operations are not shown or described in detail to avoid obscuring aspects of the invention.

While the forgoing examples are illustrative of the principles of the present invention in one or more particular applications, it will be apparent to those of ordinary skill in the art that numerous modifications in form, usage and details of implementation can be made without the exercise of inventive faculty, and without departing from the principles and concepts of the invention. Accordingly, it is not intended that the invention be limited, except as by the claims set forth below.

The verbs “to comprise” and “to include” are used in this document as open limitations that neither exclude nor require the existence of also un-recited features. The features recited in depending claims are mutually freely combinable unless otherwise explicitly stated. Furthermore, it is to be understood that the use of “a” or “an”, that is, a singular form, throughout this document does not exclude a plurality.

### INDUSTRIAL APPLICABILITY

At least some embodiments of the present invention find industrial application in mineralogy identification, analysis and mapping systems.

### ACRONYMS LIST

ASIC Application-Specific Integrated Circuit
FPGA Field-Programmable Gate Array
FTIR Fourier-Transform InfraRed spectroscopy
k-NN Nearest Neighbors (k-NN)
LIBS Laser-Induced Breakdown Spectroscope
LIDAR Light Detection and Ranging
PCA Principal Component Analysis
PLS Partial Least Squares
RAM Random-Access Memory
ROI Region of Interest
SBC Single Board Computer
UI User Interface
XRF X-ray Fluorescence
XRD X-ray Diffraction

1. A method for active hyperspectral mineralogy sensing during a Run-of-Mine in field conditions in an uncontrolled environment, wherein the method comprises:
illuminating a point on a surface of a mining material with a first broadband light signal having a range of illumination within a wavelength range of about 1350 and about 2500 nm, to enable the determination of information related to at least one water-related, carbonate-related or hydroxide-related absorbance feature;
receiving a reflected version of the first broadband light signal, wherein said reflected version of the first broadband light signal is a reflection from the mining material at different wavelengths within the range of illumination of the first broadband light signal;
determining a reflectance spectrum of the point of the mining material based on said reflected version of the first broadband light signal and further determining said information related to at least one water-related, carbonate-related or hydroxide-related absorbance feature based on the reflectance spectrum; and
performing qualitative and quantitative mineralogy analysis based on said information related to at least one water-related, carbonate-related or hydroxide-related absorbance feature.

2. The method according to claim 1, wherein said illumination of the point of the mining material is performed using a broadband light signal of at least one supercontinuum laser.

3. The method according to claim 1, further comprising:
utilizing a part of the first broadband light signal for measurement of reference measurement spectrum of said first broadband light signal before illuminating the point on the surface of the mining material; and
correcting the reflectance spectrum of the point of the mining material with the reference measurement spectrum of the first broadband light signal before performing said qualitative and quantitative mineralogy analysis.

4. The method according to claim 1, comprising:
identifying a mineral by applying a pre-calibrated machine learning classifier and the at least one water-related, carbonate-related or hydroxide-related absorbance feature in the wavelength range of the first broadband light signal.

5. The method according to claim 1, comprising:
identifying the concentration of at least one mineral by applying a pre-calibrated quantitative model and the at least one water-related, carbonate-related or hydroxide-related absorbance feature in the wavelength range of the first broadband light signal.

6. The method according to claim 1, comprising:
point-by-point scanning of a plurality of points on the surface of the mining material, according to a predetermined scanning pattern, wherein said scanning comprises illuminating each point of the mining material with the first broadband light signal and receiving said reflected version of the first broadband light signal;
determining the plurality of reflectance spectra for each point of the mining material; and
generating a hyperspectral cube for the surface of the mining material based on the combination of all reflectance spectra and corresponding coordinates according to the predetermined scanning pattern.

7. The method according to claim 6, further comprising:
identifying a mineralogical content and average mineral concentrations across the surface of the mining material by applying a pre-calibrated machine learning classifier and a pre-calibrated quantitative model to said hyperspectral cube and the at least one water-related, carbonate-related or hydroxide-related absorbance feature.

8. The method according to claim 6, further comprising:
developing a map of a mineral distribution across the surface of the mining material by applying a pre-calibrated machine learning classifier and a pre-calibrated quantitative model to said hyperspectral cube and the at least one water-related, carbonate-related or hydroxide-related absorbance feature.

9. The method according to claim 1, further comprising:
illuminating the point of the mining material with a second broadband light signal having a range within a wavelength range of about 400 and about 1100 nm, to enable the determination of information related to at least one iron-related absorbance feature;
receiving a reflected version of the second broadband light signal, wherein said reflected version of the second broadband light signal is a reflection from the point of the mining material at different wavelengths within the range of illumination of the second broadband light signal;
determining another reflectance spectrum of the point of the mining material based on said reflected version of the second broadband light signal and further determining said information related to at least one iron-related absorbance feature based on the reflectance spectrum; and
perform said hyperspectral mineralogy analysis based on said information related to at least one iron-related absorbance feature.

10. A method according to claim 9, wherein said illumination of the point of the mining material with the second broadband light signal is performed using at least one monochromatic laser.

11. An active hyperspectral mineralogy sensing system for active hyperspectral mineralogy sensing during a Run-of-Mine in field conditions in an uncontrolled environment, comprising:
at least one light source arranged to illuminate a point on a surface of a mining material with a first broadband light signal having a range of illumination within a wavelength range of about 1350 and about 2500 nm, to enable determination of information related to at least one water-related, carbonate-related or hydroxide-related absorbance feature;
at least one first spectrometer arranged to receive a reflected version of the first broadband light signal, wherein said reflected version of the first broadband light signal is a reflection from the mining material at different wavelengths within the range of illumination of the first broadband light signal; and
at least one processor configured to determine a reflectance spectrum of the point of the mining material based on said reflected version of the first broadband light signal, to determine said information related to at least one water-related, carbonate-related or hydroxide-related absorbance feature based on the reflectance spectrum and to perform mineralogy analysis based on said information related to at least one water-related, carbonate-related or hydroxide-related absorbance feature.

12. The active hyperspectral mineralogy sensing system according to claim 11, further comprising:
at least one beam splitter to separate a part of the first broadband light signal for reference measurement, wherein
the at least one first spectrometer is connected to the at least one processor and arranged to receive a reference version of the first broadband light signal, wherein said reference version of the first broadband light signal is a reflection from the beam splitter at different wavelengths within the range of illumination of the first broadband light signal.

13. The active hyperspectral mineralogy sensing system according to claim 11, further comprising at least one supercontinuum laser as the light source.

14. The active hyperspectral mineralogy sensing system according to claim 11, further comprising:
a pan-tilt unit arranged to perform point-by-point scanning of the surface of the mining material to enable further identification of the mineralogical content and average mineral concentrations across the surface of the mining material and/or mineralogical mapping of the surface of the mining material.

15. The active hyperspectral mineralogy sensing system according to claim 11, further comprising:
at least one light source arranged to illuminate the point of the mining material with a second broadband light signal having a range of illumination within a wavelength range of about 400 and about 1100 nm, to enable determination of information related to at least one iron-related absorbance feature;
at least one second spectrometer arranged to receive a reflected version of the second broadband light signal, wherein said reflected version of the second broadband light signal is a reflection from the mining material at different wavelengths within the range of illumination of the second broadband light signal; and
at least one processor configured to determine a reflectance spectrum of the point of the mining material based on said reflected version of the second broadband light signal, to determine said information related to at least one iron-related absorbance feature based on the reflectance spectrum and to perform said mineralogy analysis based on said information related to the at least one iron-related absorbance feature.

16. A non-transitory computer readable medium for active hyperspectral mineralogy sensing during a Run-of-Mine in field conditions in an uncontrolled environment, the non-transitory computer readable medium comprising instructions which, when executed by an apparatus, cause the apparatus to carry out the method according to claim 1.
